(19) United States
(12) Patent Application Publication
(10) Pub. No.: US 2017/0152534 A1
(43) Pub. Date: Jun. 1, 2017

(54) Rumenic Acid Rich Conjugated Linoleic Acid

(71) Applicant: Stepan Company, Northfield, IL (US)

(72) Inventors: Vladimir Dragan, Garfield, NJ (US); Jenifer Heydinger Galante, Oakland, NJ (US); Jos Heimerikx, Heerhugowaard (NL)

(21) Appl. No.: 15/428,339

(22) Filed: Feb. 9, 2017

Related U.S. Application Data
(63) Continuation of application No. PCT/US2015/044194, filed on Aug. 7, 2015.
(60) Provisional application No. 62/035,893, filed on Aug. 11, 2014.

(51) Int. Cl. C12P 7/64; A23L 33/12; A61K 31/201

(52) U.S. Cl. CPC C12P 7/6427; A61K 31/201; A23L 33/12; A23V 2002/00

(57) ABSTRACT

A process for preparing a conjugated linoleic acid (CLA) material that is enriched in the cis 9, trans 11 (rumenic acid) CLA isomer. The process involves subjecting a material containing at least 75 weight % CLA moieties to an enzymatic conversion, wherein the enzyme has the ability to discriminate between the cis 9, trans 11 and trans 10, cis 12 isomers. The enzyme is advantageously a lipase derived from Candida rugosa. The resulting CLA product stream is distilled to separate the free fatty acid fraction from the glyceride fraction. The recovered free fatty acid fraction contains about 55 weight % to about 70 weight % of the cis 9, trans 11 isomer (rumenic acid), and has a weight ratio of cis 9, trans 11 isomer to trans 10, cis 12 isomer of at least 3.5:1. The material enriched in rumenic acid may be used in foods, particularly infant formulas, or in food supplements or in pharmaceutical compositions.

### RELATED APPLICATIONS

This a continuation of and claims priority to PCT Patent Application PCT/US2015/44194 having an International filing date of Aug. 7, 2015, which claims priority to U.S. Provisional Application No. 62/035,893, filed Aug. 11, 2014. The contents of the applications referred to above are hereby incorporated by reference.

### FIELD OF THE INVENTION

The present technology relates in general to conjugated linoleic acid (CLA), more particularly to CLAs that are enriched in the cis 9, trans 11 CLA isomer, and have particular ratios of the cis 9, trans 11 and trans 10, cis 12 CLA isomers. The present technology is also directed to processes for preparing CLAs that have particular ratios of the cis 9, trans 11, and trans 10 cis 12 isomers.

### BACKGROUND OF THE INVENTION

Conjugated linoleic acids (CLAs) refer to a mixture of positional and geometric isomers of linoleic acids (octadecadienoic acids), which are unsaturated fatty acids considered essential to the human diet and found preferentially in dairy products and meat. CLAs have generated much interest in the academic and business communities because of their nutritional, therapeutic, and pharmacological properties. There are numerous known CLA compositions, along with various known methods to prepare and/or purify such compositions. See, for example, U.S. Pat. Nos. 6,184,009 (Cain, et al.); 7,029,691 (Saebo, et al.); and 7,514,096 (Haraldsson, et al.)

Previously known methods to produce conjugated linoleic acid materials include, for example, isomerization with an excess of alkali metal hydroxide in an aqueous or alcoholic medium, which leads to a quantitative isomerization. The resulting CLA is a mixture of positional isomers of linoleic acid. It is theoretically possible that eight geometric isomers of 9,11 and 10,12 octadecadienoic acid (cis 9, cis 11; cis 9, trans 11; trans 9, cis 11; trans 9, trans 11; cis 10, cis 12; cis 10, trans 12; trans 10, cis 12; and trans 10, trans 12) could result from the isomerization of cis 9, cis 12- octadecadienoic acid. However, the cis 9, trans 11 and trans 10, cis 12 isomers are thermodynamically favored, resulting in a higher distribution of the cis 9, trans 11 and trans 10, cis 12 isomers in the resulting CLA product. The cis 9, trans 11 and trans 10, cis 12 isomers are typically present in the resulting product in a weight ratio of about 1:1.

U.S. Pat. No. 6,184,009 to Cain, et al. describes a process for preparing a CLA material having a ratio of the cis 9, trans 11, trans 10, cis 12 isomers that is different than 1.0, such that the CLA material is enriched in one or the other of the cis 9, trans 11 or trans 10, cis 12 isomers. The process uses a lipase, such as that derived from Geotrichum candidum or Candida rugosa, to enzymatically convert the CLA material and obtain a CLA material that is enriched in one or the other of the cis 9, trans 11 or trans 10, cis 12 isomers. The '009 patent does not disclose the amounts of other CLA isomers in the resulting CLA material, nor the total amounts of saturated fatty acids or trans non-CLA fatty acids in the resulting material.

U.S. Pat. No. 5,892,074 and U.S. Pat. No. 6,153,774 describe a process for making CLA enriched in the cis 9, trans 11 isomer from ricinoleic acid by forming mesylate or tosylate esters at the 12-hydroxy position, and then reacting the diester with a strong organic base, 1,8-diazabicyclo [54.0] undec-7-ene (DBU). The sulfonyl halides used as the derivatizing agent, however, liberate corrosive hydrogen halides that must be scrubbed from the process effluent gas and neutralized. In addition, the organic byproduct resulting from treatment with the organic base is an organic amine salt of a sulfonic acid, which must be completely removed if the CLA is to be used as a food supplement. Further, the organic base is a fairly expensive organic amine.

WO 2007/070302 describes a process for preparing CLA enriched in the cis 9, trans 11 isomer from ricinoleic acid by forming a carboxylic ester at the 12-hydroxy position, and then reacting the intermediate ester with an alkoxide base. Although the process avoids the use of mesylate or tosylate derivatizing agents, the process still involves a chemical process to achieve the cis 9, trans 11 isomer that requires the removal of byproducts, such as carboxylic acid salts.

There is still a need for improved processes to produce superior CLA compositions that are enriched in the highly desired cis 9, trans 11 isomer, also known as rumenic acid, but that are also low (less than 2%, preferably less than 1% by weight) in undesirable trans, trans isomers and undesirable trans non-CLA fatty acids. Additionally, there is a need for an improved process that can economically prepare such CLA compositions without a solvent, and without the need for further purification steps to remove impurities and unwanted isomers.

### SUMMARY OF THE INVENTION

One aspect of the present technology is directed to an organic material comprising from about 60 weight % to about 95 weight % of conjugated linoleic acid moieties, wherein the moieties comprise the geometrical isomers cis 9, trans 11 and trans 10, cis 12 linoleic acid in a weight ratio of cis 9, trans 11 to trans 10, cis 12 of at least about 3.5:1, and wherein about 55 weight % to about 70 weight % of the moieties are cis 9, trans 11 linoleic acid. The linoleic acid moieties further comprise less than about 2 weight % of trans, trans conjugated linoleic acid isomers, less than about 1 weight % trans non-conjugated fatty acids, and less than about 10 weight % saturated fatty acids.

In another aspect, the present technology is directed to a nutritional composition comprising a conjugated linoleic acid component comprising from about 55 weight % to about 70 weight % cis 9, trans 11 conjugated linoleic acid, and a weight ratio of cis 9, trans 11 to trans 10, cis 12 conjugated linoleic acid isomers that is at least 3.5:1. Such compositions can be used in a food product or a nutritional supplement, and are particularly useful for maintaining or improving joint mobility.

In a further aspect, the present technology is directed to an organic material comprising from about 60 weight % to about 95 weight % of conjugated linoleic acid moieties, wherein the moieties comprise the geometrical isomers cis 9, trans 11 and trans 10 cis, 12 linoleic acid in a weight ratio of cis 9, trans 11 to trans 10, cis 12 of less than about 1, and wherein at least 40 weight % of the moieties are trans 10, cis 12 linoleic acid. The linoleic acid moieties further comprise less than about 3 weight % of trans, trans conjugated linoleic acid isomers, less than about 1 weight % trans non-conjugated fatty acids, and less than about 10 weight % saturated fatty acids.

Another aspect of the present technology is a process for preparing a conjugated linoleic acid material comprising from about 55 weight % to about 70 weight %, based on total CLA, of cis 9, trans 11 conjugated linoleic acid isomer, and having a weight ratio of cis 9, trans 11 to trans 10, cis 12 isomers of at least 3.5:1. The process comprises providing a starting material comprising geometrical isomers of conjugated linoleic acid moieties, wherein the geometrical isomers comprise cis 9, trans 11 and trans 10, cis 12 linoleic acid moieties in a weight ratio of about 1:1. The starting material is hydrolyzed with a lipase enzyme that is selective for the cis 9, trans 11 isomer to form a conjugated linoleic acid reaction stream comprising a free fatty acid fraction and a glyceride fraction. The reaction is stopped by deactivating the enzyme when the ratio of cis 9, trans 11 isomer to trans 10, cis 12 isomer is at least 5.25:1, but not more than 8.1:1. The reaction stream is then distilled to separate the free fatty acid fraction from the glyceride fraction. The recovered free fatty acid fraction comprises about 55 weight % to about 70 weight % of the desirable cis 9, trans 11 conjugated linoleic acid isomer, and has the desired weight ratio of cis 9, trans 11 to trans 10, cis 12 isomers of at least 3.5:1.

In a further embodiment, the process of the present technology includes the step of recovering the glyceride fraction from the CLA reaction stream, whereby the glyceride fraction comprises at least 40 weight % of the trans 10, cis 12 isomer and a weight ratio of cis 9, trans 11 isomer to trans 10, cis 12 isomer of less than 1. The ratio of mono- and diglycerides to triglycerides in the glyceride fraction is about 1:1.

### DETAILED DESCRIPTION

The present technology is directed to a CLA material that comprises a mixture of free fatty acids, wherein the free fatty acid mixture comprises about 55 weight % to about 70 weight % rumenic acid (cis 9, trans 11 isomer) and less than 2% by weight trans, trans CLA isomers. Desirably, the free fatty acid mixture has a weight ratio of cis 9, trans 11 isomer to trans 10, cis 12 isomer that is at least 3.5:1, preferably at least 4:1, and has less than 10% by weight saturated fatty acids, and less than 1% by weight non-CLA fatty acid. Unless the context dictates otherwise, the weight ratios and weight percents described herein are based on the weight of the CLA moiety, i.e. a polyunsaturated fatty acid having a carbon chain length of 18 carbons, without regard to whether the CLA is in the form of a free fatty acid, a fatty acid alkyl ester, a salt, or a glyceride.

The present technology is also directed to a process for preparing the CLA material enriched in rumenic acid. The process involves subjecting a material containing at least 75 weight % CLA moieties to an enzymatic conversion, wherein an enzyme is applied that has the ability to discriminate between the cis 9, trans 11 and trans 10, cis 12 isomers. One suitable enzyme is a lipase derived from Candida Rugosa which is commercially available under the tradename AY AMANO 400SD from Amano Enzyme USA Co. Ltd., Elgin, Ill.

Starting materials for the present process are those that contain at least 75 weight % of conjugated linoleic acid moieties and comprise at least the cis 9, trans 11 and trans 10, cis 12 isomers. The conjugated linoleic acid moieties can be in the form of free fatty acids, fatty acid alkyl esters, fatty acid salts, or alternatively, mono-, di-, or triglycerides, or mixtures thereof. Good results have been obtained when the conversion is performed on a triglyceride-containing material commercially available under the tradename Clarinol G80 from Stepan Lipid Nutrition, Maywood, N.J., having a ratio of cis 9, trans 11 to trans 10, cis 12 isomers of about 1:1.

The starting CLA material is obtained from a source of linoleic acid, such as fish oils or vegetable oils. Safflower oil is a particularly suitable source of linoleic acid for the starting material. The source of linoleic acid is processed by process techniques known in the art to obtain the starting CLA-containing material.

In a first step of one embodiment of the process, the triglyceride-containing material is combined with water to form a reaction mixture, and a lipase derived from Candida rugosa is added to the mixture. The amount of water is about 5 weight % to about 15 weight % based on the total weight of the reaction mixture, and the amount of lipase is about 20 to about 30 ppm of the total weight of the reaction mixture. The lipase is selective for the cis 9, trans 11 CLA isomer and selectively hydrolyzes the CLA triglycerides. The hydrolysis is conducted at a temperature of about 40° C. to 50° C., and progress of the hydrolysis is monitored by gas chromatography (GC). The hydrolysis is allowed to continue until the weight ratio of cis 9, trans 11 isomer to trans 10, cis 12 isomer is at least 5.25:1 but not more than 8.1:1. Typical reaction times for the hydrolysis reaction are about 5 to about 8 hours.

Following the hydrolysis, vacuum is applied to remove water from the reaction mixture. When vacuum reaches about 20 mmHg, the mixture is heated to at least 80° C. to deactivate the enzyme. Optionally, the vacuum pressure can be further reduced to about 5 mmHg to further dry the resulting CLA product stream without deactivating the enzyme.

The resulting product CLA stream, which contains both free fatty acids and glycerides, is then distilled by molecular distillation to separate the free fatty acid fraction from the glyceride fraction. Optionally, the CLA stream can be filtered to remove solids and/or enzymes prior to the distillation operation.

In one embodiment, distillation is accomplished by supplying the CLA stream to a wiped-film distillation apparatus or other low residence time distillation apparatus. Such a distillation apparatus minimizes the time at which the distilled stream is subject to elevated temperatures thereby preventing or at least reducing thermal rearrangement of the CLA into undesirable isomers. For example, residence times of less than 2 minutes are advantageous for minimizing the potential for thermal rearrangement of the double bonds at elevated temperatures. Temperatures for the distillation can range from about 140° C. to about 190° C. depending on the distillation equipment used. The distillation apparatus is also preferably operated at a reduced pressure, such as, for example about 0.01 mmHg to about 1 mmHg. Such low pressures are advantageous since they allow the use of lower distillation temperatures, which is important due to the thermally labile nature of the CLAs.

One example of a suitable distillation apparatus is a wiped-film evaporator supplied by Pope Scientific, Inc. (Saukville, Wis.). The wiped film evaporator has heated walls and a condenser at the center of the unit. The CLA stream to be distilled flows down the heated walls. The CLA stream is distributed over the walls by means of a wiper, which forms a film on the heated walls. A condenser is in the center of the unit, minimizing the time at which the distilled stream is at elevated temperatures. The distillate stream flows down the condenser and the residue continues to flow down the walls of the distillation unit. Both the distillate and the distillation bottoms can be cooled upon exiting the unit by means of external heat exchangers. The internal condenser allows rapid condensation and recovery of the distilled material.

One advantage of the present technology is that the distillation operation yields two entirely different, unique, and useful CLA product streams, one enriched in desirable cis 9, trans 11 isomers, and the other enriched in desirable trans 10, cis 12 isomers. The overhead distillate stream resulting from the wiped-film distillation is the free fatty acid fraction and comprises from about 55 weight % to about 70 weight % rumenic acid (cis 9, trans 11 CLA) and less than 10 weight % glycerides. The bottom distillation stream from the distillation is the glyceride fraction and comprises at least 40 weight % trans 10, cis 12 CLA isomer content and less than about 10 weight % free fatty acids. Advantageously, the process of the present technology is accomplished without a solvent, the use of which can require additional processing steps in order to remove it. The isomer composition of the resulting CLA product streams can be determined by GC, as is known in the art.

In addition to separating the free fatty acid fraction from the glyceride fraction, the distillation operation substantially removes non-conjugated trans fatty acids and unwanted CLA isomers, such as the trans, trans isomers, from each of the product fractions without the need for further purification steps to remove the unwanted impurities and isomers. The resulting free fatty acid CLA product has less than about 2 weight %, alternatively less than about 1 weight % of undesirable trans, trans isomers, and less than about 1 weight % trans non-conjugated fatty acids. The resulting CLA glyceride product has less than about 3 weight %, alternatively less than about 2 weight % of undesirable trans, trans isomers, and less than about 1 weight % trans non-conjugated fatty acids. The glyceride product also has a weight ratio of mono- and diglyceride to triglyceride of about 1:1.

The CLA materials of the present technology have a variety of uses. These include, for example, the reduction of body fat in animals; increasing muscle mass in animals; reducing body weight in humans; attenuating allergic reactions in animals; preventing weight loss due to immune stimulation in animals; elevating CD-4 and CD8 cell counts in animals; increasing the mineral content of bone in animals; preventing skeletal abnormalities in animals; decreasing the amount of cholesterol in the blood of animals, and maintaining or increasing joint mobility. In each case the term “animal” includes all mammals, including humans. As used herein, “joint mobility” includes, but is not limited to, joint function, joint strength, and/or range of motion. Joint strength can be assessed, for example, via a hand dynamometer, and range of motion can be assessed, for example, via a goniometer.

The CLA materials of the present technology may be incorporated into animal feeds, nutritional supplements, dietary applications, or pharmaceutical applications. The CLA materials may be formulated with suitable carriers such as starch, sucrose or lactose in tablets, pills, dragees, capsules, solutions, liquids, slurries, suspensions and emulsions. They may also be provided in aqueous solution or oily solution. The tablet, pill or capsule comprising the CLA may be coated with an enteric coating which dissolves at a pH of about 6.0 to 7.0. A suitable enteric coating which dissolves in the small intestine, but not in the stomach is cellulose acetate phthalate. The isomer enriched CLA may also be provided by any of a number of other routes, including, but not limited to, intravenous, intramuscular, intra-arterial, intramedullary, intrathecal, intraventricular, transdermal subcutaneous, intraperitoneal, intranasal, enteral, topical, sublingual or rectal means. Further details on techniques for formulation and administration may be found in the latest edition of Remington's Pharmaceutical Sciences (Maack Publishing Co., Easton, Pa.).

The CLA materials of the present technology may also be provided as a supplement in various prepared food products and drinks. For the purposes of this application, prepared food product means any natural, processed, diet or non-diet food product to which the CLA material of the present technology has been added. The CLA material may be incorporated into various prepared food products, including, but not limited to diet drinks, diet bars, supplements, prepared frozen meals, candy, snack products (e.g. chips), prepared meat products, milk, cheese, yogurt and any other fat or oil containing foods.

One particular contemplated use of the CLA material enriched in rumenic acid is in an infant formula. Human breast milk typically has a weight ratio of the cis 9, trans 11 to trans 10, cis 12 CLA isomers of about 4:1. By adjusting the processing parameters or, alternatively, by mixing higher ratio CLA with commercially available 1:1 isomer ratio CLA, a free fatty acid fraction can be obtained having the desired 4:1 weight ratio of cis 9, trans 11 to trans 10, cis 12 isomers. The fatty acids can be converted into glycerides prior to use in the infant formula.

One particular contemplated use of the CLA glyceride material enriched in the trans 10, cis 12 isomer is in personal care and cosmetic applications, particularly topical skin applications. The glyceride material has a ratio of mono- and diglycerides to triglycerides of about 1:1, which has been found to provide good emulsification properties useful for personal care applications.

### EXAMPLES

### Example 1

A triglyceride material (Clarinol G80 available from Stepan Lipid Nutrition) containing 78.7% of conjugated linoleic acid (CLA), of which 37.3% was the cis 9, trans 11 isomer, and 37.4% was the trans 10, cis 12 isomer, is used as the starting material. A lipase solution is prepared by mixing 3.2 g Candida rugosa lipase (Amano AY 400 DS available from Amano Enzyme USA Co. Ltd.) with 691 g of water for 1 hour at 40° C. to 45° C. 681 g of this solution, containing 0.46% weight of lipase, is combined with 111.7 kg of the starting triglyceride material and 12.3 kg of distilled water to form a reaction mixture. The reaction mixture is adjusted to 40° C. to 45° C. After 5 to 6 hours of reaction time, the progress of the reaction is monitored by GC. When the ratio of cis 9, trans 11 isomer to trans 10, cis 12 isomer reaches at least 5.25:1, vacuum is applied to the reaction mixture to remove water. When the vacuum pressure reaches 20 mmHg, the reaction is stopped by heating to 80° C. to 85° C. for at least one hour to deactivate the enzyme. The vacuum is reduced to 5 mmHg to further dry the resulting CLA stream.

The CLA product stream is fed to a wiped-film distillation apparatus to separate the free fatty acid fraction from the triglyceride fraction. Separation of the fractions is based on differences in their vapor pressure. The overhead stream is the free fatty acid fraction and the bottoms stream is the triglyceride fraction. Analysis by GC shows that the free fatty acid fraction contains 74.6% CLA (59.1% cis 9, trans 11 isomer and 14.5% trans 10, cis 12 isomer), with a weight ratio of cis 9, trans 11 to trans 10, cis 12 isomer of 4.08. As a percentage of total CLA, the cis 9, trans 11 isomer is 79%. A full analysis of the free fatty acid fraction is shown in Table 1.

Analysis of the glyceride fraction shows that the fraction contains 80% CLA (28.9% cis 9, trans 11 isomer and 46.9% trans 10, cis 12 isomer), with a weight ratio of cis 9, trans 11 isomer to trans 10, cis 12 isomer of 0.6. As a percentage of total CLA, the trans 10, cis 12 isomer is 59%. A full analysis of the glyceride fraction is shown in Table 1.

TABLE 1

| | Free Fatty Acid Fraction | Glyceride |
|---|---|---|
| | (Rumenic Acid Rich CLA) | Fraction |
| FFA | 96.2 | 6.03 |
| Monoglyc | 3.1 | 5.8 |
| Diglyc | 1.1 | 44.5 |
| Triglyc | <0.5 | 41.4 |
| Polymers | <0.5 | 2.3 |
| Total Glycerides | 4.2 | 91.7 |
| GC: | | |
| C16:0 | 6.09 | 4.87 |
| C18:0 | 0.73 | 1.95 |
| C18:1 | 16.20 | 10.70 |
| C18:2, linoleic | 1.11 | 0.70 |
| total saturated | 7.25 | 7.53 |
| Total trans non-CLA | 0.57 | 0.63 |
| c9,t11-CLA | 59.10 | 28.90 |
| t10,c12-CLA | 14.50 | 46.90 |
| tr,tr-CLA | 0.45 | 2.12 |
| Other CLA | 0.55 | 2.08 |
| Total Isomers | 73.6 | 75.8 |
| Total CLA | 74.6 | 80.0 |
| Rumenic as % Total CLA | 79 | |
| Rumenic as % Isomers | 80 | |
| Ratio 9,11:10,12 | 4.08 | |
| 10,12 as % Total CLA | | 59 |
| 10,12 as % Isomers | | 62 |
| Ratio 10,12:9,11 | | 1.62 |

The presently technology is now described in such full, clear, concise, and exact terms as to enable any person skilled in the art to which it pertains, to practice the same. It is to be understood that the foregoing describes preferred embodiments of the invention and that modifications may be made therein without departing from the spirit or scope of the invention as set forth in the appended claims.

1. An organic material comprising from about 60 weight % to about 95 weight % of conjugated linoleic fatty acid moieties, wherein the moieties comprise the geometrical isomers cis 9, trans 11 and trans 10, cis 12 linoleic acid in a weight ratio of cis 9, trans 11 to trans 10, cis 12 of at least 3.5:1, wherein about 55 weight % to about 70 weight % of the moieties are cis 9, trans 11 linoleic acid, and wherein less than about 2 weight % of the moieties comprise trans, trans conjugated linoleic acid isomers.

2. The organic material of claim 1, wherein the organic material comprises a mixture of free fatty acids and contains less than about 10 weight % glycerides.

3. The organic material of claim 1, wherein the organic material comprises less than about 10 weight % of saturated fatty acids.

4. The organic material of claim 1, wherein the organic material comprises less than about 1 weight % of trans non-conjugated fatty acids.

5. A nutritional composition for a human or animal diet, the composition comprising:
a conjugated linoleic acid component comprising from about 55 weight % to about 70 weight % cis 9, trans 11 conjugated linoleic acid, and a weight ratio of cis 9, trans 11 to trans 10, cis 12 conjugated linoleic acid isomers of at least 3.5:1.

6. The nutritional composition of claim 5, wherein the composition is a food product.

7. The nutritional composition of claim 5, wherein the composition is a nutritional supplement.

8. The nutritional composition of claim 5, wherein the conjugated linoleic acid component is present in an amount effective to maintain or improve joint mobility.

9. The nutritional composition of claim 6, wherein the food product is an infant formula.

10. An organic material comprising from about 60 weight % to about 95 weight % of conjugated linoleic acid moieties, wherein the moieties comprise the geometrical isomers cis 9, trans 11 and trans 10, cis 12 linoleic acid in a weight ratio of cis 9, trans 11 to trans 10, cis 12 of less than about 1, wherein at least 40 weight % of the moieties are trans 10, cis 12 linoleic acid, and wherein less than about 3 weight % of the moieties comprise trans, trans conjugated linoleic acid isomers.

11. The organic material of claim 10, wherein the organic material comprises a mixture of glycerides, and contains less than about 10 weight % free fatty acids.

12. The organic material of claim 11, wherein the mixture of glycerides comprises mono-, di-, and triglycerides in a weight ratio of mono- and diglyceride to triglyceride of about 1:1.

13. The organic material of claims 10, wherein the organic material comprises less than about 10 weight % saturated fatty acids.

14. The organic material of claim 10, wherein the organic material comprises less than about 1 weight % of trans non-conjugated fatty acids.

15. A process for the preparation of a conjugated linoleic acid material enriched in cis 9, trans 11 conjugated linoleic acid isomer and having a particular weight ratio of cis 9, trans 11 to trans 10, cis 12 isomers, the process comprising:
providing a starting material comprising geometrical isomers of conjugated linoleic acid moieties, wherein the geometrical isomers comprise cis 9, trans 11 and trans 10, cis 12 linoleic acid moieties in a weight ratio of about 1:1;
hydrolyzing the starting material with a lipase enzyme that is selective for the cis 9, trans 11 isomer to form a conjugated linoleic acid reaction stream that comprises a free fatty acid fraction and a glyceride-fraction;
distilling the conjugated linoleic acid reaction stream by molecular distillation to separate the free fatty acid fraction and the glyceride fraction; and
recovering the free fatty acid fraction, whereby the free fatty acid fraction has a weight ratio of the cis 9, trans 11 to trans 10, cis 12 isomers of at least 3.5:1, and about 55 weight % to about 70 weight % of the free fatty acid fraction comprises cis 9, trans 11 linoleic acid.

16. The process of claim 15, wherein the glyceride fraction is recovered following the distillation step, whereby the glyceride fraction has a weight ratio of the cis 9, trans 11 to trans 10, cis 12 isomers of less than 1, and at least 40 weight % of the glyceride fraction comprises trans 10, cis 12 linoleic acid.

17. The process of claim 15, wherein the lipase is derived from Candida Rugosa.

18. The process of claim 15, wherein the free fatty acid fraction comprises about 60 weight % to about 95 weight % conjugated linoleic acid moieties and contains less than about 10 weight % glycerides.

19. The process of claim 16, wherein the glyceride fraction comprises 60 weight % to about 95 weight % conjugated linoleic acid moieties and contains less than about 10 weight % free fatty acids.

20. The process of claim 15, wherein the starting material is hydrolyzed during the hydrolyzing step until the conjugated linoleic acid reaction stream has a weight ratio of cis 9, trans 11 isomer to trans 10, cis 12 isomer of at least 5.25:1, but not more than 8.1:1.

21. The process of claim 20, wherein, following the hydrolysis step, vacuum is applied to the reaction stream.
